(19) United States
(12) Patent Application Publication
(10) Pub. No.: US 2021/0022743 A1
(43) Pub. Date: Jan. 28, 2021

(54) IN VIVO CROSSLINKING OF EMBOLIC HYDROGELS USING BIOORTHOGONAL CLICK CHEMISTRY

(71) Applicant: Boston Scientific Scimed, Inc., Maple Grove, MN (US)

(72) Inventors: Joseph Thomas Delaney, JR., Minneapolis, MN (US); Ajay Gupta, Shoreview, MN (US); Daniel K. Tomaschko, Savage, MN (US); Tatyana Dyndikova, Minneapolis, MN (US)

(21) Appl. No.: 16/935,963

(22) Filed: Jul. 22, 2020

Related U.S. Application Data
(60) Provisional application No. 62/877,137, filed on Jul. 22, 2019.

(51) Int. Cl. A61B 17/12; C08J 3/075; C08J 3/24; A61K 31/787

(52) U.S. Cl. CPC A61B 17/1214; C08J 3/075; A61B 2017/00526; A61K 31/787; A61B 17/12113; C08J 3/242

(57) ABSTRACT

A crosslinked embolic hydrogel is disclosed, the crosslinked embolic hydrogel comprising a hydrophilic polymer functionalized with first reactive groups and a crosslinking agent functionalized with second reactive groups; wherein the first and second reacting groups comprise a biorthogonally reactive pair that react to form the crosslinked embolic hydrogel. Methods and systems are also disclosed.

This application claims the benefit of U.S. Provisional Application No. 62/877,137, filed Jul. 22, 2019, the content of which is herein incorporated by reference in its entirety.

### FIELD OF THE INVENTION

The present disclosure relates generally to crosslinked embolic hydrogels, methods, and systems.

### BACKGROUND

Numerous embolization products have been developed, both for interventional oncology, as well as treatment of aneurysms. These embolization products often include coils that can be delivered relatively easily into large blood vessels, but which often do not embolize large vessels efficiently or completely. Alternatively, embolic plugs have been created for large vessels, but can be difficult to deliver. Similarly, for smaller vessels or arterio-venous malformations (AVMs) it can be difficult to embolize using either coils or plugs due to the small and/or complex nature of the embolization target. Therefore, a need exists for embolization improvements, both for large and small blood vessels.

### SUMMARY

This disclosure is directed, in a first aspect, to a crosslinked embolic hydrogel, the crosslinked embolic hydrogel is formed from a hydrophilic polymer functionalized with first reactive groups and a crosslinking agent functionalized with second reactive groups; wherein the first and second reactive groups comprise biorthogonally reactive pairs that react to form the crosslinked embolic hydrogel.

In a second aspect, in addition to one or more of the preceding or following aspects, or in the alternative to some aspects, the first or second reactive groups include a plurality of amine groups, acid groups, and combinations thereof.

In a third aspect, in addition to one or more of the preceding or following aspects, or in the alternative to some aspects, either the first or second reactive groups include an azide group.

In a fourth aspect, in addition to one or more of the preceding or following aspects, or in the alternative to some aspects, either the first or second reactive groups include an alkyne, tetrazine, fluorosydnones, or combinations thereof.

In a fifth aspect, in addition to one or more of the preceding or following aspects, or in the alternative to some aspects, either the first or the second reactive groups include a strained alkyne.

In a sixth aspect, in addition to one or more of the preceding or following aspects, or in the alternative to some aspects, the first reactive groups and second reactive groups form a covalent bond when brought in contact with each other.

In a seventh aspect, in addition to one or more of the preceding or following aspects, or in the alternative to some aspects, the first and second reactive groups form a tri-azole ring upon reacting.

In an eighth aspect, in addition to one or more of the preceding or following aspects, or in the alternative to some aspects, either the hydrophilic polymer or the crosslinking agent are retained on an embolization coil.

In a ninth aspect, in addition to one or more of the preceding or following aspects, or in the alternative to some aspects, either the hydrophilic polymer or crosslinking agent are retained on microbeads.

In a tenth aspect, in addition to one or more of the preceding or following aspects, or in the alternative to some aspects, the hydrophilic polymer, the crosslinking agent, or both include branched polymers.

In an eleventh aspect, in addition to one or more of the preceding or following aspects, or in the alternative to some aspects, the hydrophilic polymer, the crosslinking agent, or both include non-branched polymers.

In a twelfth aspect, in addition to one or more of the preceding or following aspects, or in the alternative to some aspects, each of the hydrophilic polymer and crosslinking agent include at least two reactive groups.

In a thirteenth aspect, a method for forming a crosslinked embolic hydrogel is disclosed, the method includes providing a hydrophilic polymer functionalized with first reactive groups; and providing a crosslinking agent functionalized with second reactive groups; combining the hydrophilic polymer with the crosslinking agent such that the first and second reactive groups bound form a crosslinked embolic hydrogel.

In a fourteenth aspect, in addition to one or more of the preceding or following aspects, or in the alternative to some aspects, either the first or second reactive groups include a plurality of amine groups, acid groups, and combinations thereof.

In a fifteenth aspect, in addition to one or more of the preceding or following aspects, or in the alternative to some aspects, either the first or second reactive groups include an azide group, an alkyne, tetrazine, fluorosydnones, or combinations thereof.

In a sixteenth aspect, in addition to one or more of the preceding or following aspects, or in the alternative to some aspects, either the first or the second reactive groups include a strained alkyne.

In a seventeenth aspect, in addition to one or more of the preceding or following aspects, or in the alternative to some aspects, the first and second reactive groups form a tri-azole ring upon reacting.

In an eighteenth aspect, in addition to one or more of the preceding or following aspects, or in the alternative to some aspects, either the hydrophilic polymer or the crosslinking agent are retained on an embolization coil.

In a nineteenth aspect, in addition to one or more of the preceding or following aspects, or in the alternative to some aspects, either the hydrophilic polymer or crosslinking agent are retained on microbeads.

In a twentieth aspect, a system for forming a crosslinked embolic hydrogel, the system having a hydrophilic polymer functionalized with first reactive groups; and a crosslinking agent functionalized with second reactive groups; the first and second reactive groups include a biorthogonally reactive pair.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE DRAWINGS

The present subject matter may be more completely understood and appreciated in consideration of the following detailed description of various embodiments in connection with the accompanying drawings.

FIG. 1 is a schematic representation of example orthogonally reactive groups joined to polymer or crosslinker materials.

FIG. 2 is a schematic representation of a first branched polymeric material showing first orthogonally reactive groups.

FIG. 3 is a schematic representation of a second branched polymeric material showing second orthogonally reactive groups.

FIG. 4A is a schematic representation of first and second polymeric materials of FIG. 2 and FIG. 3 reacted together to form a hydrogel.

FIG. 4B is a closeup representation of a portion of the hydrogel of FIG. 4A.

FIG. 5 is a schematic representation of a non-branched polymeric material showing orthogonally reactive groups.

FIG. 6 is a schematic representation of a microbead having reactive groups secured to it.

FIG. 7 is a schematic representation of a kidney tumor being treated with microbeads to create an embolic seal.

FIG. 8 is a schematic representation of a peripheral occlusion device formed using a coil coated with a crosslinkable hydrogel.

FIG. 9 is a schematic representation of an aneurysm treated with a coil coated with a crosslinkable hydrogel to create an embolic seal.

While embodiments herein are susceptible to various modifications and alternative forms, specifics thereof have been shown by way of example and drawings and will be described in detail. It should be understood, however, that the scope herein is not limited to the particular examples described. On the contrary, the intention is to cover modifications, equivalents, and alternatives falling within the spirit and scope herein.

### DETAILED DESCRIPTION

This disclosure is directed, in a first aspect, to a crosslinked embolic hydrogel. the crosslinked embolic hydrogel is formed from a hydrophilic polymer functionalized with first reactive groups and a crosslinking agent functionalized with second reactive groups; wherein the first and second reactive groups comprise biorthogonally reactive pairs that react to form the crosslinked embolic hydrogel. Thus, the biorthogonally reactive pairs selectively react with one another to form the hydrogel.

In certain embodiments a two-part injectable in vivo crosslinking hydrogel is formed. In an example embodiment, a hydrophilic polymer is functionalized with biorthogonally reactive end groups and is made into a dilute aqueous solution. A crosslinking agent with corresponding biorthogonally reactive groups is also made. When the two are sequentially injected into the arterial vasculature of a patient they combine in the smaller blood vessel to form a crosslinked hydrogel, blocking blood flow. The hydrophilic polymer and crosslinking agent react to form a gel only where the two components combine with each other in concentrations high enough to form a crosslinked network (the gel point). Due to the bioorthogonality of the reaction, neither component will typically substantially react with anything else in the body other than its counterpart, offering high levels of chemo selectivity.

The hydrophilic polymers and crosslinking agents may have, for example, a branched or linear architecture, including stars, dendrites, combs, etc. Generally the average reactive functionality between the components is 2 or greater. In some implementations the average reactive functionality is greater than 2, greater than 3, greater than 4, or greater than 5. Optionally the average reactive functionality is less than 10, less than 9, less than 8, less than 7, or less than 6. In certain implementations the average functionality is from 2 to 10, from 3 to 8, from 4 to 7, or from 5 to 6. It will be understood that in some implementations, especially for large reactive bodies, the average functionality can be greater than 10.

The reactive groups can be, for example, an azide, alkyne, tetrazine, fluorosydnones, or combinations thereof. An azide group is particularly appropriate because it is small, metabolically stable, and does not naturally exist in cells. Thus, azide groups do not have major competing biological side reactions. An alkyne group is not as small, but it still has significant stability and bioorthogonality. Specific biorthogonal click pairings include, for example, strain-promoted azide-alkyne (SPAAC) click reactions; inverse electron-demand Diels-Alder (iEDDA) conjugations using tetrazine and either transcyclooctene, norbornene, or cyclopropane; and nitrile oxides to strained alkenes.

In some implementations either a hydrophilic polymer with a first reactive group or a crosslinking agent with a first reactive group is bonded to an embolic coil. The coil can be delivered so that it anchors in the target vessel and partially blocks the vessel. Thereafter a liquid containing the counterpart reactive group (either a crosslinking agent or a hydrophilic polymer with a second reactive group) is added. The first and second reactive groups react until gelling and embolization occurs.

It is alternatively possible to coat microbeads with a first reactive group so as to reduce the “set-up” time for forming the embolization and/or reduce the amount of hydrogel that must be formed. The presence of the microbeads reduces the volume of space to be filled by the hydrogel, and as such less hydrogel is needed. In an example embodiment the microbeads containing one reactive group of a reactive pair are delivered to a target (such as a cancerous tumor) and then a material (such as a hydrogel or other crosslinking agent) comprising the other reactive group of the reactive pair is added to the same location to crosslink the microbeads. In this manner the microbeads can be incorporated into a hydrogel and/or crosslinked with one another. Microbeads can be administered by way of a catheter or other application device. In an example embodiment microbeads are administered into a target zone via a catheter, and thereafter a crosslinking agent is administered via the catheter to bind the beads to one another and form a hydrogel securing the beads in place, thereby embolizing a target location.

In some implementations the microbeads are all the same size, while in other implementations the microbeads vary in size. For example, relatively large microbeads can be administered along with relatively small microbeads that can occupy the space between the relatively large microbeads, reducing the amount of hydrogel necessary. Also, microbeads can be administered in conjunction with other biorthogonal oligomers. For example, for smaller vessels alternate injections of small microbeads containing a first reactive group on the surface followed with injections of a second reactive group including biorthogonal oligomers molecules to bind microbeads together until desired embolization is achieved.

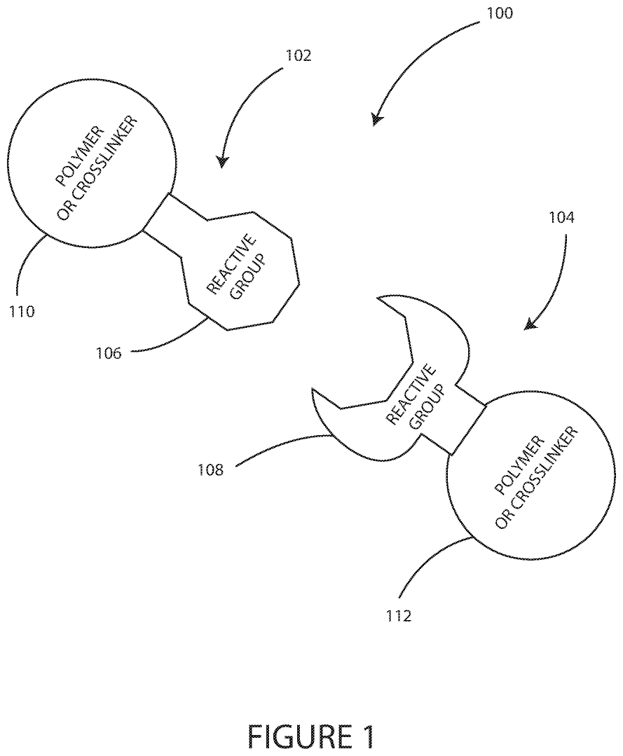

Referring now to the figures, FIG. 1 is a schematic representation of biorthogonally reactive pairs secured to polymers and/or crosslinking agent. The biorthogonally reactive pair 100 includes a first reactive composition 102 and a second reactive composition 104. First reactive composition 102 includes a first reactive group 106 and is secured to a base material 110, such as a polymer (for example a hydrophilic polymer) or a crosslinker agent. The second reactive composition 104 includes a second reactive group 108 that is secured to a different base 112, such as a polymer or crosslinking agent. The first reactive compositions 102 and second reactive composition 104 are shown schematically, and it will be appreciated that the figure does not show the chemical structure or relative size of the molecules or reactive groups.

In actual practice the base materials 110, 112, such as a hydrophilic polymer or a crosslinker agent, are typically much larger than the reactive groups 106, 108. Examples of suitable base materials 110, 112 include, as mentioned above, various polymer components or various crosslinker components. The base materials 110, 112 can be joined, using the click chemistry described herein, to form an embolic material, such as an embolic material to cut off blood flow to cancerous tumors, or an embolic material to fill an aneurism. The base material, besides being a polymeric composition, can include a substrate such as microbeads, coils, tubes or similar substrates. The use of microbeads, for example, is beneficial because the microbeads themselves will partially fill an area to embolized, and as such less of other materials are needed. In use, the microbeads are delivered to a target (such as a cancerous tumor) and then a material forming a reactive pair is added to the same location to crosslink with the microbeads. In this manner the microbeads can be incorporated into a hydrogel and/or crosslinked with one another. Similarly, an embolization coil used to fill a volume (such as an aneurism) can be coated with one part of a reactive pair. A material, typically a polymer with two or more matching reactive pairs on each molecule, is then delivered to the location where the coil has been placed, thereby crosslinking and filling gaps in the embolization coil. In this manner the relatively precise deliverability of the embolization coil is combined with the precise, localize reaction with a crosslinking material to form a seal that is less porous than the embolization coil alone.

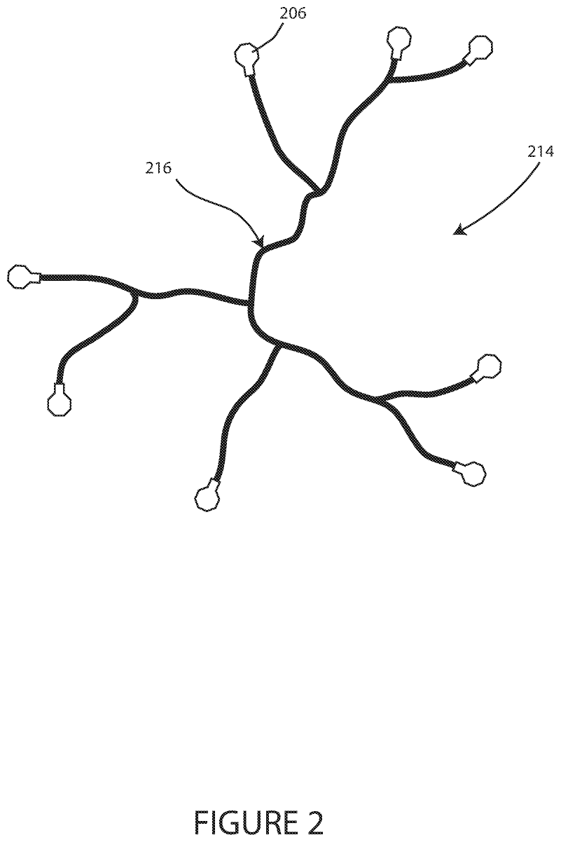

The reactive groups 106, 108 are selected so as to be biorthogonally reactive such that they readily react only with one another. However, when they are brought in contact with one another they readily form a covalent bond, which then binds the polymer or crosslinking agents 110, 112 to one another. In the representation shown in FIG. 1 the base materials 110, 112 are shown as each containing only one reactive group 106, 108. Generally each base material 110, 112 will be secured to more than one reactive group 106, 108 so as to promote crosslinking of materials, not just reaction of materials. The presence of multiple reactive groups 106, 108 (as discussed below with regard to FIG. 2) provides for a more robust crosslinking and gelling result. Multiple reactive groups 106, 108 are also beneficial because they compensate for some reactive groups that do not react, such as those that are sterically hindered, while still allowing crosslinking to occur with the remaining reactive groups. It will be appreciated, however, that in some situations only one reactive group 106, 108 is present, such as when the reactive groups are secured to a larger substrate, such as a microbead, in which case a multitude of reactive groups secured to the microbead performs similar to a very large polymer by allowing crosslinking between microbeads to occur or by allowing microbeads to be secured by covalent bonds within a hydrogel.

FIG. 2 is a schematic representation of a first branched polymeric material 214 showing first biorthogonally reactive groups 206 secured to a polymeric backbone 216. This figure is shown for representative purposes, and it will be understood that the polymeric backbone 216 can have multiple configurations. It will be appreciated that the polymeric material 214 can have (for example) a branched or linear architecture, including stars, dendrites, combs, etc. The reactive groups 206 are not reactive with one another, but rather primarily reactive only with biorthogonally reactive counterparts on a separate molecule. The two pairs of a biorthogonally reactive pair are typically not located on the same polymer otherwise they would react with themselves. The location of the reactive groups 206 can vary depending upon the type and size of the polymeric backbone 216, such as being located at the end of branches of the backbone 216, along the backbone 216 itself between the end of branches, or at both the ends of the branches and along the backbone.

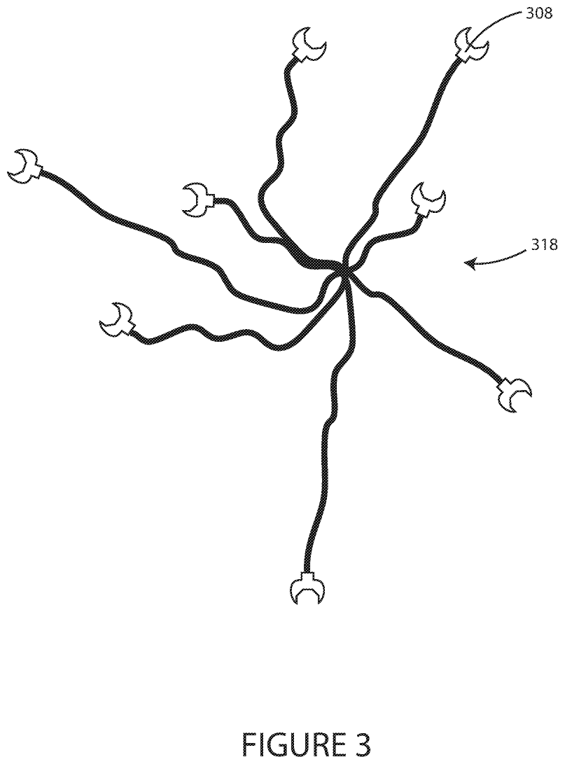

FIG. 3 is a schematic representation of a second branched polymeric material 318 showing second orthogonally reactive groups 308. As was the case with reactive groups 206 of FIG. 2, reactive groups 308 are not reactive with one another, but rather primarily reactive only with biorthogonally reactive counterparts on a separate molecule. Thus, the polymeric material 214 of FIG. 2 can react with the polymeric material 318 of FIG. 3, but reactive groups 206 and polymeric material 214 do not react with themselves, and polymeric material 318 and reactive groups 308 do not react with themselves.

In the constructions shown in FIG. 2 and FIG. 3 the two polymer backbones, despite being schematic representations, are shown as approximately the same size, and as having the same approximate number of reactive groups. In some embodiments the two polymeric materials forming the two biorthogonal polymeric back bones will be approximately the same size, the same shape, and have approximately the same number of reactive groups. However, in other implementations the two polymer backbones will have different sizes, different shapes, and different numbers of reactive groups. For example, in an embodiment, a first component having a first reactive group will be on a much larger polymer backbone than a second component having a second reactive group. This difference in size can be used, for example, when it is desirable to place the first component in a target zone (such as the blood supply of a tumor or an aneurism), and to then administer a second component on smaller polymeric backbone. The benefit of the smaller polymeric backbone can be to promote penetration of the second smaller component deeper into the target zone so as to reach (and react with) as much of the first component as possible.

The shape of the polymeric backbones can also be selected to obtain desirable results. In some implementations a branching backbone is desired (such as shown in FIG. 2), in other implementations a star shaped construction is desired (such as shown in FIG. 3). Although these two constructions show multiple reactive groups on complex branched polymers, in the alternative the polymer can be unbranched and have just two reactive groups in an example construction. Further, it will be understood that multiple polymeric constructions can be used at once, such as having a first reactive group on a variety of different polymeric constructions (straight, star, branched, etc.). The variety of polymeric constructions can be beneficial in applications where voids of a variety of shapes and sizes are desired, such as blocking the blood vessels serving a tumor, in which case the vessels can vary substantially in size.

In addition, in some constructions it is desirable to have a greater number of a first reactive group than of a second reactive group. This can be true, for example, when it is particularly desirable that all of the second reactive group be bonded. By having an excess of the first reactive group the chances of binding to a high proportion of the second reactive group is increased. Thus, in many implementations the first and second reactive groups will be generally or approximately equal to one another, but in some implementations one reactive group will be at least 25 percent more common, at least 50 percent more common, at least 75 percent more common, at least 100 percent more common, at least 200 percent more common, or at least 300 percent more common than a second reactive group.

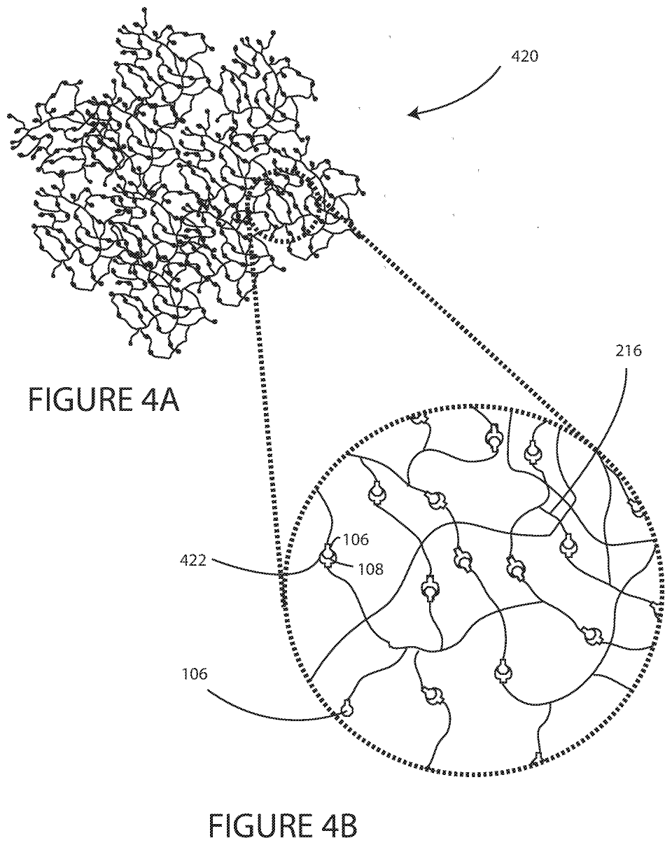

FIG. 4A is a schematic representation of first and second polymeric materials of FIG. 2 and FIG. 3 reacted together to form a hydrogel 420. FIG. 4B is a closeup representation of a portion of the hydrogel of FIG. 4A, showing reacted pairs 422 formed of first reactive group 106 and second reactive group 108. It will be appreciated that in typical circumstances there will be some non-reacted groups, such as a non-reacted first reactive group 106 shown in FIG. 4B. As noted above, the relative ratio of first and second reactive groups 106, 108 will often be close to 1:1, but in certain embodiments one of the reactive groups 106, 108 will be more common than the other, such as when various application details and/or geometries mean that only a portion of one of the reactive groups is likely to react, or where full reaction of one of the groups is particularly desired (in which case higher numbers of the other reactive pair is desired).

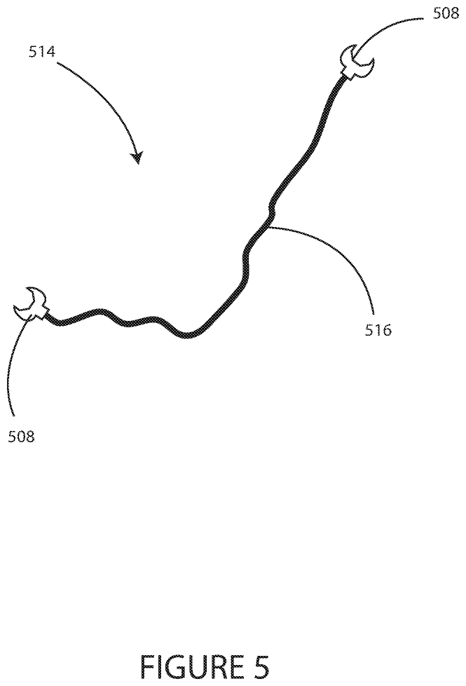

FIG. 5 is a schematic representation of a non-branched polymeric material 514 showing orthogonally reactive groups 508 secured to a polymeric backbone 516. Typically the polymeric material 514 will have at least two reactive groups 508 so as to permit crosslinking. The reactive groups can account for a small or large portion of the overall polymeric material 514. For example, the molecular weight of the reactive groups can account for, as an example, greater than one percent of the total polymeric material, greater than two percent of the total polymeric material, greater than five percent of the total polymeric material; or greater than ten percent of the total polymeric material.

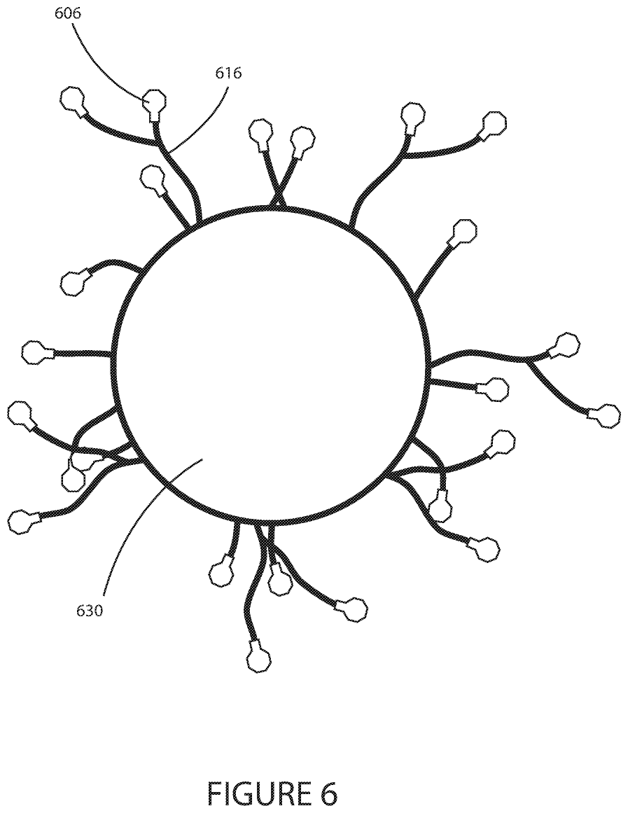

FIG. 6 is a schematic representation of a microbead 630 having orthogonally reactive groups 606 secured to the beads, such as by polymeric backbones 616. This schematic representation is not intended to be drawn to scale, and is only a functional representation of the various components. It will be appreciated that the reactive groups 606 can be secured to the microbead 630 without a polymeric backbone, thus directly to microbead 630. However, in such cases the crosslinking agent (not shown) must be long enough to bridge between microbeads 630 and ideally between multiple microbeads 630.

The microbeads can have, for example, a diameter from about 10 microns to 1,000 microns (1 millimeter), optionally less than 900 microns, less than 800 microns, less than 700 microns, less than 600 microns, less than 500 microns, less than 400 microns, less than 300 microns, less than 200 microns or less than 100 microns. In some embodiments the microbeads are less than 90 microns, less than 80 microns, less than 70 microns, less than 60 microns, less than 50 microns, less than 40 microns, less than 30 microns, or less than 20 microns.

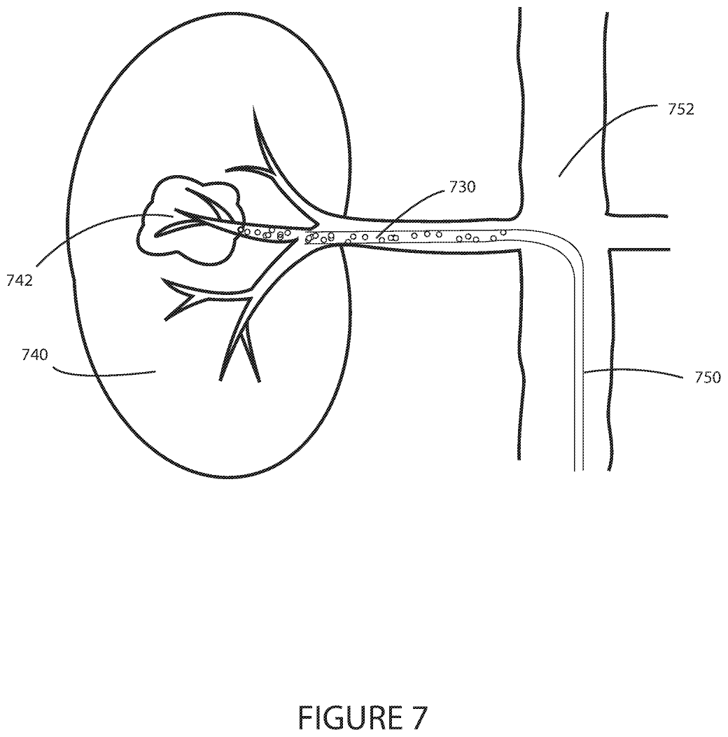

FIG. 7 is a schematic representation of a kidney 740 with a tumor 742 being treated with microbeads 730 to create an embolic seal. Microbeads 730 can be administered by way of a catheter 750 that extends through the descending aorta 752 to the renal artery and then into smaller renal arteries. The microbeads 730 are administered into a target zone, and thereafter a crosslinking agent is administered to bind the beads to one another and form a hydrogel securing the beads in place and embolizing small target renal arteries. The microbeads flow into various narrowing blood vessels, but crosslinking of the microbeads or incorporating them into a hydrogel provides improved sealing and reduction of blood flow.

The microbeads can all be of the same size or be of different sizes. Also the microbeads can all be delivered at once or delivered over time, but typically the microbeads will be delivered in a first stage followed by delivery of a crosslinking agent that is generally not administered as part of a microbead. However, both materials can be administered by microbead in some embodiments, such as situations where a first microbead is administered followed by a much smaller second microbead that is able to penetrate deeper into the deposit of the first microbeads. Also, it is possible to use a combination of microbeads and non-microbeads to deliver reactive materials, such as by having a first reactive group on microbeads but also on polymeric materials not secured to a micro bead. In this manner the second reactive group binds the beads to one another and to the polymeric materials.

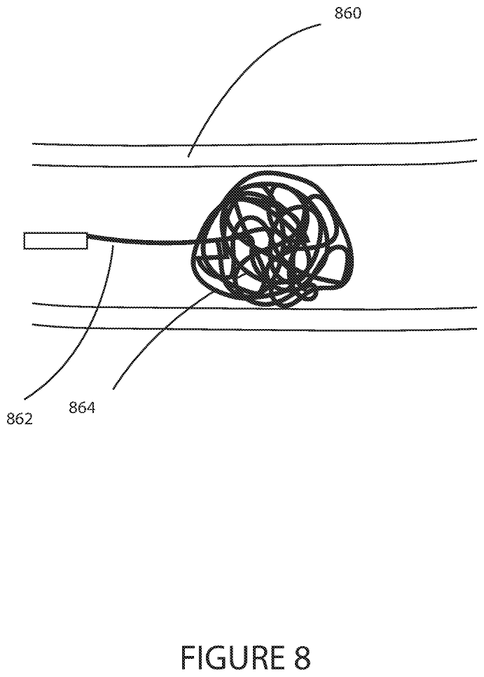

FIG. 8 is a schematic representation of a cross section of blood vessel 860 with a peripheral occlusion device formed from a coil 864, such as an embolization coil, coated with a crosslinkable hydrogel (not shown). Various constructions can be implemented, but in a typical embodiment the combination of the coil 864 itself along with a hydrogel that is formed by in vivo crosslinking forms a rapid and effective embolism. In an example embodiment the coil 864 comprises a flexible substrate, such as a non-reactive bio-compatible metal material coated with one of a pair of biorthogonal reactive groups. The biorthogonal reactive groups can be directly secured to the flexible substrate, bounded to a polymeric backbone that is in turn secured to the flexible substrate, or otherwise secured thereto. Upon placement within a target zone a crosslinking material is added having the other pair of the biorthogonal reactive group. The biorthogonal reactive pairs react to form a hydrogel surrounding the substrate, such as a coil. In this manner the coil is more readily stabilized, but also the open areas between the coil strands are filled with hydrogel, and gaps around the perimeter of the coil (adjacent to a blood vessel wall, for example) are filled in with hydrogel.

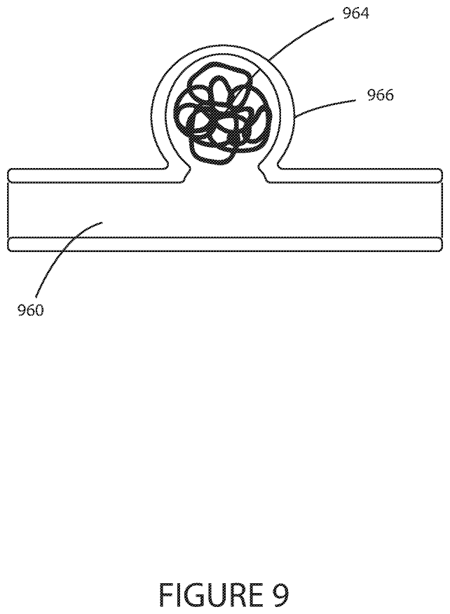

Similarly, FIG. 9 is a schematic representation of an aneurysm 966 being treated with a coil 964 to create an embolism in a blood vessel 960. As is the case with the occlusion device of FIG. 9, in a typical embodiment the combination of the coil 964 itself along with a hydrogel that is formed by in vivo crosslinking forms a rapid and effective embolism. In an example embodiment the coil 964 comprises a flexible substrate, such as a non-reactive bio-compatible metal material coated with one of a pair of biorthogonal reactive groups. The biorthogonal reactive groups can be directly secured to the flexible substrate, bounded to a polymeric backbone that is in turn secured to the flexible substrate, or otherwise secured thereto. Upon placement within a target zone a crosslinking material is added having the other pair of the biorthogonal reactive group. The biorthogonal reactive pairs react to form a hydrogel surrounding the substrate, such as a coil. In this manner the coil is more readily stabilized, but also the open areas between the coil strands are filled with hydrogel, and gaps around the perimeter of the coil (adjacent to a blood vessel wall, for example) are filled in with hydrogel.

It should be noted that, as used in this specification and the appended claims, the phrase “configured” describes a system, apparatus, or other structure that is constructed to perform a particular task or adopt particular characteristics. The phrase “configured” can be used interchangeably with other similar phrases such as “arranged”, “arranged and configured”, “programmed” “constructed and arranged”, “constructed”, “manufactured and arranged”, and the like.

All publications and patent applications in this specification are indicative of the level of ordinary skill in the art to which the present technology pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive.

We claim:

1. A crosslinked embolic hydrogel, the crosslinked embolic hydrogel formed from:
a hydrophilic polymer functionalized with first reactive groups; and
a crosslinking agent functionalized with second reactive groups;
wherein the first reactive groups and second reactive groups comprise a biorthogonally reactive pair that react to form the crosslinked embolic hydrogel.

2. The crosslinked embolic hydrogel of claim 1, wherein either the first or second reactive groups comprise a plurality of amine groups, acid groups, and combinations thereof.

3. The crosslinked embolic hydrogel of claim 1, wherein either the first or second reactive groups comprise an azide group.

4. The crosslinked embolic hydrogel of claim 1, wherein either the first or second reactive groups comprise an alkyne, tetrazine, fluorosydnones, or combinations thereof.

5. The crosslinked embolic hydrogel of claim 1, wherein either the first or the second reactive groups comprise a strained alkyne.

6. The crosslinked embolic hydrogel of claim 1, wherein the first reactive groups and second reactive groups form a covalent bond when brought in contact with each other.

7. The crosslinked embolic hydrogel of claim 1, wherein the first and second reactive groups form a tri-azole ring upon reacting.

8. The crosslinked embolic hydrogel of claim 1, wherein either the hydrophilic polymer or the crosslinking agent are retained on an embolization coil.

9. The crosslinked embolic hydrogel of claim 1, wherein either the hydrophilic polymer or crosslinking agent are retained on microbeads.

10. The crosslinked embolic hydrogel of claim 1, wherein the hydrophilic polymer, the crosslinking agent, or both comprise branched polymers.

11. The crosslinked embolic hydrogel of claim 1, wherein the hydrophilic polymer, the crosslinking agent, or both comprise non-branched polymers.

12. The crosslinked embolic hydrogel of claim 1, wherein each of the hydrophilic polymer and crosslinking agent comprise at least two reactive groups.

13. A system for forming a crosslinked embolic hydrogel, the system comprising:
a hydrophilic polymer functionalized with first reactive groups; and
a crosslinking agent functionalized with second reactive groups;
wherein the first and second reactive groups comprise a biorthogonally reactive pair.

14. A method for forming a crosslinked embolic hydrogel, the method comprising:
providing a hydrophilic polymer functionalized with first reactive groups; and
providing a crosslinking agent functionalized with second reactive groups;
combining the hydrophilic polymer with the crosslinking agent such that the first and second reacting groups bound to form a crosslinked embolic hydrogel.

15. The method for forming a crosslinked embolic hydrogel of claim 14, wherein either the first or second reactive groups comprise a plurality of amine groups, acid groups, and combinations thereof.

16. The method for forming a crosslinked embolic hydrogel of claim 14, wherein either the first or second reactive groups comprise an azide group or groups, an alkyne group or groups, a tetrazine group or groups, a fluorosydnone group or groups, or combinations thereof.

17. The method for forming a crosslinked embolic hydrogel of claim 14, wherein either the first or the second reactive groups comprise a strained alkyne.

18. The method for forming a crosslinked embolic hydrogel of claim 14, wherein the first and second reactive groups form a tri-azole ring upon reacting.

19. The method for forming a crosslinked embolic hydrogel of claim 14, wherein either the hydrophilic polymer or the crosslinking agent are retained on an embolization coil.

20. The method for forming a crosslinked embolic hydrogel of claim 14, wherein either the hydrophilic polymer or crosslinking agent are retained on microbeads.
